(19) United States
(12) Patent Application Publication
(10) Pub. No.: US 2012/0029523 A1
(43) Pub. Date: Feb. 2, 2012

(54) Guide pin positioner

(76) Inventors: Andrew S. Kam, Odessa, FL (US); John X. Xerogeanes, Atlanta, GA (US); Hugh S. West, Salt Lake City, UT (US)

(21) Appl. No.: 12/804,813

(22) Filed: Jul. 29, 2010

(51) Int. Cl. A61B 17/56

(52) U.S. Cl. 606/96

(57) ABSTRACT

A guide for a pin for proper placement for guiding a reamer to make a bone tunnel for femoral ACL/PCL reconstruction features a base ring with spaced struts to allow visual access to the native ligament stump. Alternatively the base ring can be supported by a see-through structure. The ring can be circular where it contacts the bone or elliptical depending on the relation of the plane in which the distal surface of the ring is disposed and the axis of the cannulated tool body. The dimensional differences between and inner and outer surface of the base ring allows visualization of two different dimensions for a proposed tunnel location.

### FIELD OF THE INVENTION

This invention relates to surgical instruments used in arthroscopic surgical procedures to guide instruments into the proper position of a surgical worksite. More particularly, the invention relates to guides for positioning drills or guide pins through portals in arthroscopic ligament reconstruction surgical procedures.

### BACKGROUND OF THE INVENTION

Surgical procedures for arthroscopic repair of torn tendons, ligaments or other soft tissue are well known. One such common repair involves the replacement of one of the cruciate ligaments of the knee. There are some such procedures where a surgeon must select the proper location at which to drill bone tunnels adapted to receive graft ligaments such as bone-tendon-bone or soft tissue ligament constructs.

The subject invention is useful in positioning such bone tunnels on the femur at locations which approximate the anatomic location of the ACL or PCL. Other guides are known which position such bone tunnels. The issue that they present is an inability to obtain a visual observation of the site where the pin will be drilled as the hub of the guide obstructs this view when placed at the femoral attachment point of the ruptured ligament against the medial aspect of the lateral femoral condyle.

FIG. 1 shows an existing guide made by Arthrex Inc. of Naples, Fla. that features a hollow shaft 10 having a through passage 12 for positioning the hub 14 located at the distal end. The surgeon places the head against the superior inner wall of the medial femoral condyle by manipulating the proximal end 16. Passage 12 continues through hub 14 to the bone contact surface 18. The surface 18 is in a plane that intersects the axis of the passage 12 at an oblique angle with the intent that the angled orientation will put surface 18 flush with the bone when advanced from the incision and properly rotated on its axis for flush engagement. A spike 20 is located off center from the passage 12 as shown in FIG. 2. The spike 20 holds the shaft steady as a pin 22 is drilled into the bone 24 as shown in FIG. 4. The problem with the design is that the hub 14 is a solid mass and prevents the surgeon from seeing the precise location where the pin 22 will engage the proximal cortical surface of the knee and to readily see where the edges of the tunnel that will be drilled as a drill or reamer is advanced over the guide pin and into the bone. The use of this tool as a guide in double bundle PCL reconstruction makes the angle of surface 18 with respect to a plane perpendicular to the axis of passage 12 smaller or less oblique whereas a more oblique angle for a single tunnel ACL procedure allows a better approximation of the femoral ACL footprint so that an elliptical shape is created at the tunnel where the ratio of the long to the short dimension is about 1.5 to 2.

Also generally related to the present invention are U.S. Pat. Nos. 5,211,647; 5,425,733; 5,350,383; 6,629,977; 6,716,234 and 6,875,216.

The present invention addresses the issue of visual access to the site where the guide pin will enter the bone and the location and angle of the reamer as it makes the tunnel. This is accomplished with an open structure of a base ring supported with spaced struts that can be disposed in a perpendicular plane to the axis of the passage through the tool body or askew. The base ring can be circular or elliptical and when elliptical can be skewed with the axis of the tool body so as to give visual indication of the bone structure at the entrance of the tunnel to be made by the reamer. Alternatively the base ring and support structure of struts with gaps or a solid taper can also provide visual access by being clear plastic, for example. Those and other features of the present invention will be more readily apparent to those skilled in the art from a review of the detailed description and the associated drawings while recognizing that the full scope of the invention is to be found in the appended claims.

### SUMMARY OF THE INVENTION

A guide for a pin for proper placement for guiding a reamer to make a bone tunnel for femoral ACL/PCL reconstruction features a base ring with spaced struts to allow visual access to the native ligament stump. Alternatively the base ring can be supported by a see-through structure. The ring can be circular where it contacts the bone or elliptical depending on the relation of the plane in which the distal surface of the ring is disposed and the axis of the cannulated tool body. The dimensional differences between the inner and outer surface of the base ring allows visualization of two different dimensions for a proposed tunnel location and allows for providing predictable tunnel aperture geometry.

### BRIEF DESCRIPTION OF THE DRAWINGS

FIG. 1 is a side view of a prior art guide showing the solid distal hub that blocks visualization of the native ligament stump;

FIG. 2 is an end view of FIG. 1;

FIG. 3 is a perspective view of the instrument of FIG. 1 adjacent the bone;

FIG. 4 is an extreme close up view of the instrument of FIG. 3 adjacent the bone;

FIG. 5 is a side view of the preferred embodiment of the present invention that enables visualization of the native ligament stump for setting of a guide pin thereon;

FIG. 6 is an end view of FIG. 5;

FIG. 7 is a perspective view of the instrument of the present invention juxtaposed next to a bone and showing also a close up view;

FIG. 8 is an alternative view of the instrument of FIG. 5.

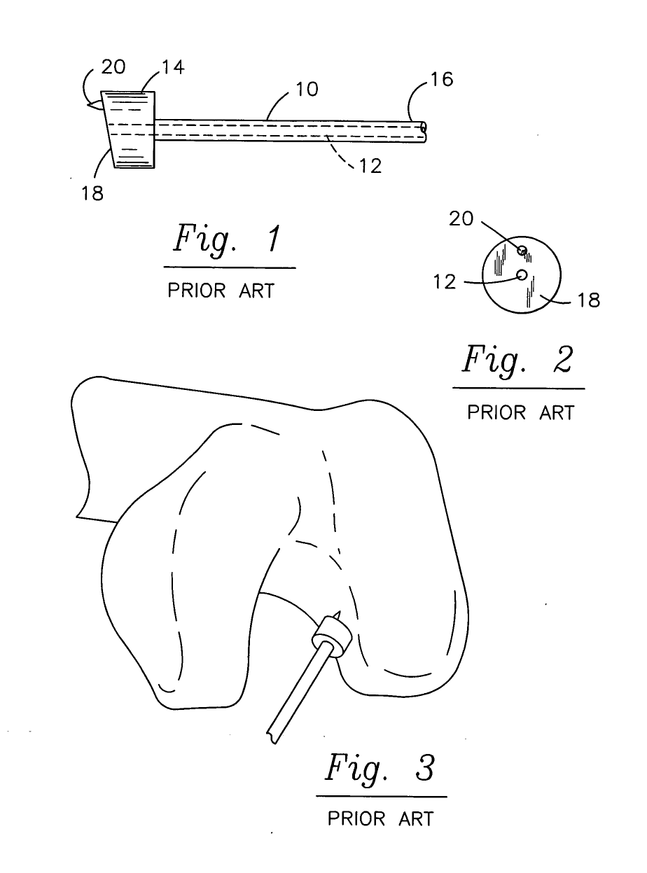

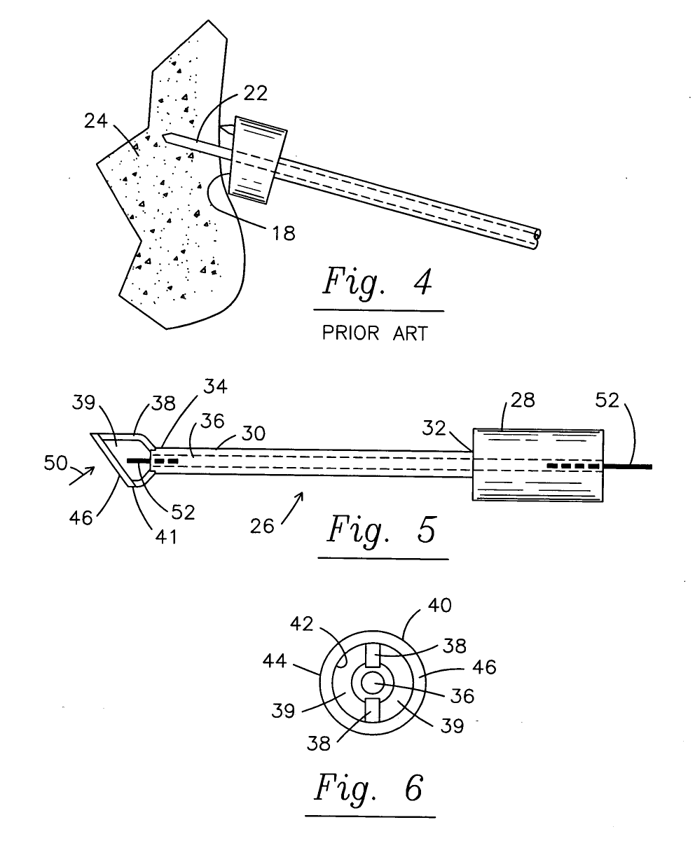

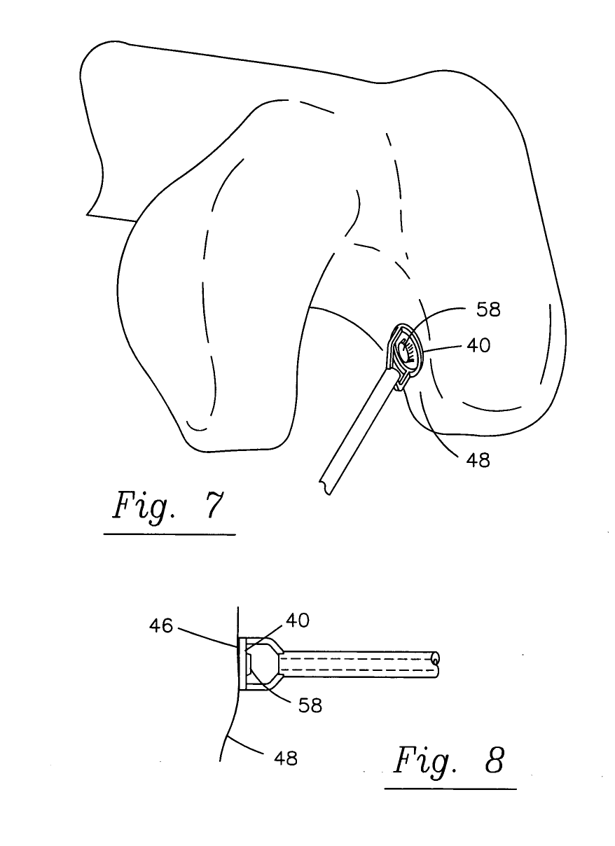

### DETAILED DESCRIPTION OF THE PREFERRED EMBODIMENT

FIG. 5 shows the instrument 26 that has a handle 28 at its proximal end with an elongated hollow shaft 30 that extends from the proximal end 32 to a distal end 34. Axial passage 36 extends from the distal end 34 back through the handle 28 and is designed to slidably receive pin 52. A plurality of struts 38 form fenestrations 39 and hold a base ring 40 away from the distal end 34. Struts 38 and fenestrations 39 form a see-through structure 41 which is annular and tapered to connect the smaller diameter of shaft end 34 to the layer size of ring 40. Ring 40 has an inner surface 42 and an outer surface 44 and a slanted contact surface 46 that engages the bone 48. Surface 46 is disposed in a plane oriented at a predetermined angle to the axis of the shaft 30. In the preferred embodiment the angle is about 30 to 45 degrees. When viewed in the direction of arrow 50 the shape defined by the inner surface 42 is elliptical with preferably the ratio of the long dimension to the short dimension being greater than about 1.1 or more depending on the angle of surface 46 as discussed above.

Significantly, with the base ring 40 being supported in an open structure of spaced apart struts 38 that can be plastic or metal there is not only an ability to see where the pin 52 will actually locate but the ring 40 using inside surface 42 or outside surface 44 allows a view of the perimeter of the tunnel that will be formed with a reamer (not shown) that will be guided over the pin 52. Alternatively to the struts 38 and fenestrations 39, the supporting structure can be a solid frustoconical shape that is clear such as plastic so that the surgeon can have visual access of the center of the tunnel as determined by the pin 52 anchor location as well as the tunnel orientation when made by the reamer.

Looking at the bottom view or end view of FIG. 6 it is possible to see passage 36 of the shaft 30. The support structure 38 that can be spaced struts or a continuous transparent structure, allows the base ring 40 to stand off from the shaft 30 so that the ring 40 can be placed squarely over the native ligament stump 58. The angled orientation of the surface 46 allows it to sit squarely or flush against the bone structure. Additionally, surface 46 can be roughened to hold it steady against the bone structure as the pin is advanced.

In some procedures there is no need to skew the plane of surface 46 with respect to a plane perpendicular to the axis of the passage 36 and in those cases the ring 40 is circular rather than elliptical. In either case the surface 46 is spaced apart from the distal end 34 of the passage 36 so that the intervening structure allows a line of sight to the ring 40 and the ligament stump 58 so that the pin 52 will be drilled at the appropriate starting location on an axis that is suitable for the reamer to create the tunnel for the ligament graft.

The above description is illustrative of the preferred embodiment and many modifications may be made by those skilled in the art without departing from the invention whose scope is to be determined from the literal and equivalent scope of the claims below.

We claim:

1. An endoscopic guide positioner comprising:
an elongated shaft having a passage therethrough;
a base structure supported from said shaft and spaced apart from a distal end of said shaft to allow visual access to said base structure.

2. The guide of claim 1 wherein:
said base structure comprises a ring.

3. The guide of claim 2 wherein:
said ring is circular.

4. The guide of claim 2 wherein:
said ring is elliptical.

5. The guide of claim 4 wherein:
the ratio of a long axis to a short axis of said elliptical ring is greater than 1 to 1.

6. The guide of claim 2 wherein:
said ring is supported from said shaft by spaced apart members that allow viewing within said ring.

7. The guide of claim 2 wherein:
said ring is supported by a see-through structure.

8. The guide of claim 7 wherein:
said structure comprises an annular tapered shape.

9. The guide of claim 8 wherein:
said tapered shape is made of a single material.

10. The guide of claim 9 wherein:
said tapered shape is made of plastic or metal.

11. The guide of claim 3, wherein:
said ring comprises a distal face disposed substantially parallel to a plane traversing an axis of said passage at about 90 degrees.

12. The guide of claim 4, wherein:
said ring comprises a distal face disposed at an angle of about 30-45 degrees to a plane traversing an axis of said passage at about 90 degrees.

13. The guide of claim 2 wherein:
said ring having a surface roughening on a distal face thereof.

14. The guide of claim 1 wherein:
said shaft further comprises a handle on a proximal end thereof with said handle providing an extension of said passage.

15. The guide of claim 5 wherein:
said ring is supported from said shaft by spaced apart strut members that allow viewing within said ring.

16. The guide of claim 5 wherein:
said ring is supported by a see-through structure.

17. The guide of claim 3 wherein:
said ring is supported from said shaft by spaced apart strut members that allow viewing within said ring.

18. The guide of claim 3 wherein:
said ring is supported by a see-through structure.
